(19) United States
(12) Patent Application Publication
(10) Pub. No.: US 2022/0273489 A1
(43) Pub. Date: Sep. 1, 2022

(54) SYSTEMS AND METHODS FOR DIFFUSE ENDOLUMINAL THERMAL LIQUID TREATMENT

(71) Applicant: Intuitive Surgical Operations, Inc., Sunnyvale, CA (US)

(72) Inventors: Robert L. Barry, Kirkland, WA (US); Erik M. Henne, Seattle, WA (US)

(21) Appl. No.: 17/623,453

(22) PCT Filed: Jul. 7, 2020
(86) PCT No.: PCT/US2020/041071

§ 371 Date: Dec. 28, 2021

Related U.S. Application Data
(60) Provisional application No. 62/871,569, filed on Jul. 8, 2019.
(60) Provisional application No. 62/871,677, filed on Jul. 8, 2019.
(60) Provisional application No. 62/871,678, filed on Jul. 8, 2019.
(60) Provisional application No. 62/938,614, filed on Nov. 21, 2019.
(60) Provisional application No. 62/988,299, filed on Mar. 11, 2020.

(51) Int. Cl. A61F 7/12; A61F 7/00; A61B 34/30; A61N 5/06; A61B 1/00; A61B 1/267; A61B 17/12

(52) U.S. Cl. CPC A61F 7/12; A61F 7/0085; A61B 34/30; A61N 5/0625; A61B 1/00165; A61B 1/2676; A61B 17/12104; A61F 2007/126; A61F 2007/0059; A61B 2034/303; A61N 2005/0604; A61N 2005/063; A61F 2007/009; A61B 2090/3933

(57) ABSTRACT

A system may comprise a liquid source from which a liquid is delivered, and a catheter coupled to the liquid source. The catheter may include a distal portion from which the liquid is released into an anatomic lumen. The system may also include an occlusion device coupled to the catheter and configured to prevent flow of the liquid in the anatomic lumen proximally of the occlusion device. The system may also include a heating device near the distal portion of the catheter. The heating device may be configured to heat the liquid to a temperature of less than a vaporization temperature for the liquid.

### CROSS-REFERENCE TO RELATED APPLICATIONS

This application claims the benefit of U.S. Provisional Application 62/871,569 filed Jul. 8, 2019; U.S. Provisional Application 62/871,677 filed Jul. 8, 2019; U.S. Provisional Application 62/871,678 filed Jul. 8, 2019; 62/938,614 filed Nov. 21, 2019; and U.S. Provisional Application 62/988,299 filed Mar. 11, 2020, all of which are incorporated by reference herein in their entirety.

This application incorporates by reference in its entirety PCT Application (Docket No. P02302-WO) filed Jul. 7, 2020, titled “Systems and Method for Localized Endoluminal Thermal Liquid Treatment.”

### FIELD

Examples described herein are related to systems and methods for diffuse endoluminal thermal treatment of diseased anatomy.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. In some examples, a minimally invasive medical instrument may be a thermal energy treatment instrument for use within an endoluminal passageway of a patient anatomy.

### SUMMARY

The following presents a simplified summary of various examples described herein and is not intended to identify key or critical elements or to delineate the scope of the claims.

In some embodiments, a system may comprise a liquid source from which a liquid is delivered, and a catheter coupled to the liquid source. The catheter may include a distal portion from which the liquid is released into an anatomic lumen. The system may also include an occlusion device coupled to the catheter and configured to prevent flow of the liquid in the anatomic lumen proximally of the occlusion device. The system may also include a heating device near the distal portion of the catheter. The heating device may be configured to heat the liquid to a temperature of less than a vaporization temperature for the liquid.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A is a simplified diagram of a patient anatomy according to some examples.

FIG. 1B illustrates a region of the patient anatomy of FIG. 1A according to some examples.

FIG. 1C is a cross-sectional view of a region of the patient anatomy of FIG. 1A according to some examples.

FIG. 2 is a flowchart illustrating a method for applying a thermal energy treatment to an endoluminal passageway, according to some examples.

FIG. 3A is a detailed view of a portion of the patient anatomy of FIG. 1A with a treatment instrument within an anatomic lumen, according to some examples.

FIG. 3B is a detailed view of a portion of the patient anatomy of FIG. 1A with a treatment instrument treating an anatomic lumen with a heated diffuse liquid, according to some examples.

FIG. 4 illustrates a treatment instrument treating an anatomic lumen with a heated diffuse liquid, according to some examples.

FIG. 5 illustrates a distal portion of a treatment instrument according to some examples.

FIG. 6 illustrates a distal portion of a treatment instrument according to some examples.

FIG. 7 illustrates a distal portion of a treatment instrument according to some examples.

FIG. 8 illustrates a distal portion of a treatment instrument according to some examples.

FIG. 9 is a flowchart illustrating a method for applying a thermal energy treatment to an endoluminal passageway, according to some examples.

FIG. 10 illustrates a distal portion of a treatment instrument according to some examples.

FIG. 11 illustrates a distal portion of a treatment instrument according to some examples.

FIG. 12A illustrates a portion of a patient anatomy exposed to an oscillating magnetic field from a magnetic field generator according to some embodiments.

FIG. 12B illustrates a distal portion of a treatment instrument according to some examples.

FIG. 13A illustrates treatment system according to some examples.

FIG. 13B illustrates a cross sectional view of a medical instrument of FIG. 13B according to some embodiments.

FIG. 14 illustrates a distal portion of a treatment instrument according to some examples.

FIG. 14B illustrates a cross sectional view of the medical instrument of FIG. 14A according to some examples.

FIG. 15 is a flowchart illustrating a method for applying a thermal energy treatment to an endoluminal passageway, according to some examples.

FIG. 16 is a flowchart illustrating a method for applying a thermal energy treatment to an endoluminal passageway, according to some examples.

FIG. 17 illustrates a distal portion of a treatment instrument according to some examples.

FIG. 18 is a flowchart illustrating a method for applying a thermal energy treatment to an endoluminal passageway, according to some examples.

FIG. 19 is a flowchart illustrating a method for occluding an artery with access via an adjacent bronchial passageway, according to some examples.

FIG. 20 illustrates an arterial occlusion device according to some examples.

FIG. 21 illustrates an arterial occlusion device according to some examples.

FIG. 22 illustrates an arterial occlusion device according to some examples.

FIG. 23 illustrates an arterial occlusion device according to some examples.

FIGS. 24A and 24B illustrate an arterial occlusion device according to some examples.

FIG. 25 illustrates a robot-assisted medical system according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The technology described herein provides techniques and treatment systems for endoluminal thermal treatment of diseased tissue. Although the examples provided herein may refer to treatment of lung tissue and pulmonary disease, it is understood that the described technology may be used in treating artificially created lumens or any endoluminal passageway or cavity, including in a patient trachea, colon, intestines, stomach, liver, kidneys and kidney calices, brain, heart, circulatory system including vasculature, fistulas, and/or the like. In some examples, treatment described herein may be referred to as endobronchial thermal liquid treatment and may be used in procedures to treat lung tumors and/or chronic obstructive pulmonary disease (COPD).

Lung tumors may include ground glass opacity tumors, semi-solid tumors, or spiculated tumors. Often, they may occur in the peripheral, outer third of the lung volume. Some lung tumor treatment methods and systems may be particularly suited for the low-density, peripheral areas of the lung. Tumors may be cancerous, and effective treatment of lung cancer may include the destruction of the tumor core, peripheral tumors, and cancerous or non-cancerous areas at the margin of the tumors. Conventional ablation treatment may directly heat the tumor core without treating the tissue at the margin. Some endoluminal thermal liquid treatments may cause infarction of a segment of the lung, thus destroying tumors and the tissue around the tumor within the infarcted region.

Chronic obstructive pulmonary disease may include one or more of a plurality of disease conditions or states including chronic bronchitis, emphysema, and bronchiectasis. Chronic bronchitis is the inflammation of bronchial tubes and may be characterized by increased mucous secretions and goblet cell hyperplasia. Emphysema is a condition in which the parenchyma including the alveoli at the distal ends of the bronchial tubes are damaged, thereby causing hyperinflation and reduced lung function. Bronchiectasis is a condition in which the bronchial tubes become widened and thickened by scarring. Pockets form in the bronchial tube walls filled with bacterial biofilm, creating a nidus for bronchiectasis exacerbations.

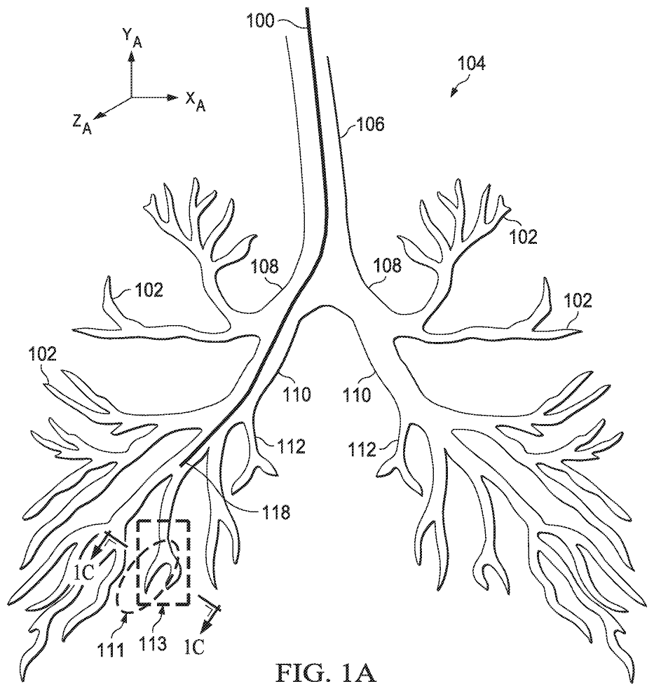

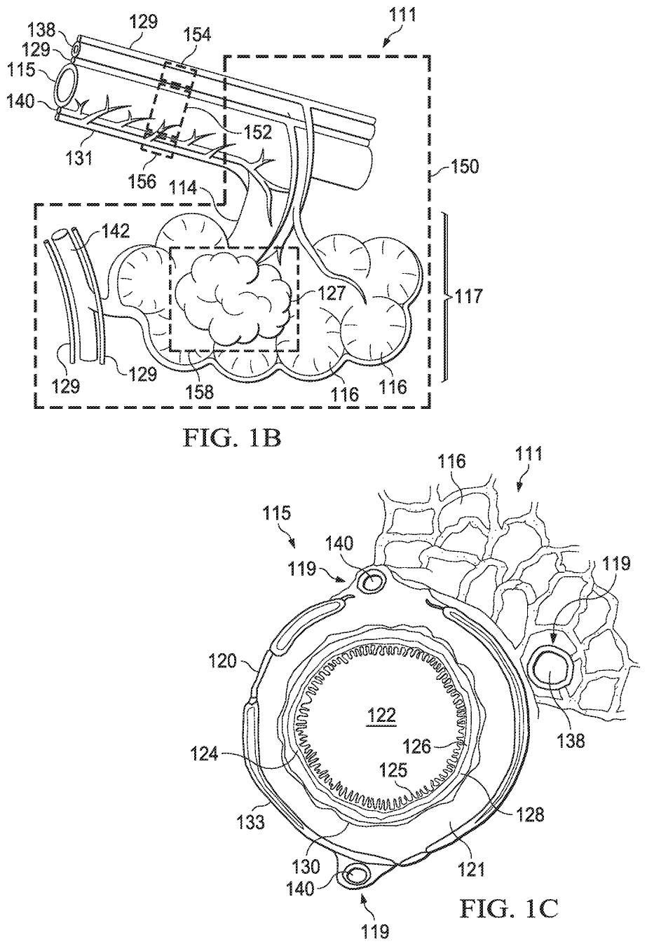

FIG. 1A. illustrates an elongated medical instrument system 100 extending within branched anatomic passageways or airways 102 of an anatomical structure 104. In some examples the anatomic structure 104 may be a lung and the passageways 102 that include the trachea 106, primary bronchi 108, secondary bronchi 110, and tertiary bronchi 112. The anatomic structure 104 has an anatomical frame of reference (XA, YA, ZA). A distal end 118 of the medical instrument 100 may be advanced into an anatomic opening (e.g. the mouth) and through the anatomic passageways 102 to perform a medical procedure, such as a thermal energy treatment, at or near target tissue located in a region 113 of the anatomic structure 104. As shown in FIG. 1B, a distal-most region 111 of the branched anatomic passageways, which may be distal of the tertiary bronchi 112, may include a small bronchus 115 and the lung parenchyma 117, including bronchioles 114 and alveoli 116, which is involved in gas exchange. Vasculature 119 extending along the bronchus 115 may include a pulmonary artery 138 and a bronchial artery 140. A pulmonary vein 142 transfers oxygenated blood from the lungs to the heart. Nerves 131 and lymphatic vessels 129 may also extend within the region 111. Target tissue 127, such as a tumor, may be formed in the parenchyma 117.

FIG. 1C illustrates a cross-sectional view of the bronchus 115 that includes a lumen 122 defined by an inner bronchial wall 124. The inner diameter of the bronchial wall 124 may be lined by an epithelium layer 126 which includes goblet cells. Mucocilia 125 are tiny hairs that extend from the epithelium 126 into the lumen 122. The epithelium 126 may be surrounded by a lamina propria layer 128 which may be surrounded by a smooth muscle layer 130. A sub mucosa layer 121 surrounds the smooth muscle layer 130, and a layer of continuous or discontinuous cartilage 133 covers the sub mucosa layer. A connective tissue layer of adventitia 120 may surround and provide support to the bronchus 115. The vasculature including the bronchial artery 140, the pulmonary artery 138, and the pulmonary vein 142 may extend along the bronchus 115 to supply blood flow to and from the lung region.

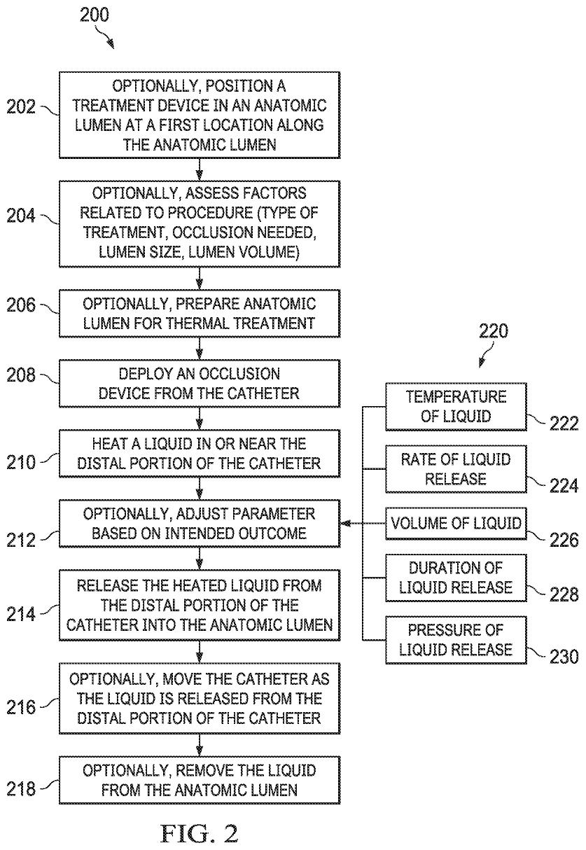

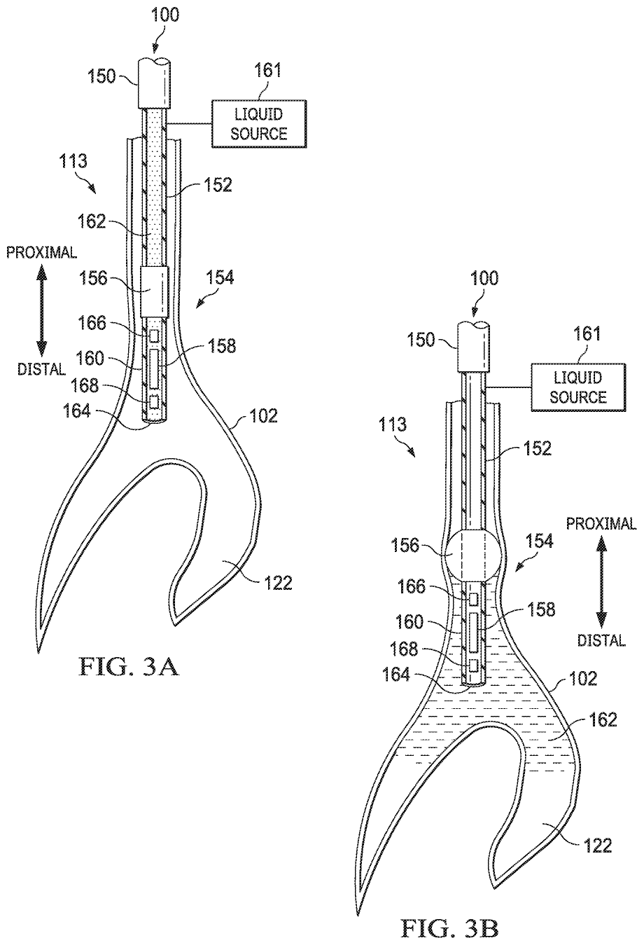

FIG. 2 is a flowchart illustrating a method 200 for applying a thermal energy treatment to an endoluminal passageway. The endoluminal thermal liquid treatment methods described in this specification may be illustrated as a set of operations or processes that may be performed in the same or in a different order than the order shown. One or more of the illustrated processes may be omitted in some embodiments of the method. Additionally, one or more processes that are not expressly illustrated may be included before, after, in between, or as part of the illustrated processes. In some embodiments, one or more of the processes of illustrated methods may be implemented, at least in part, by a control system executing code stored on non-transitory, tangible, machine-readable media that when run by one or more processors (e.g., the processors of a control system) may cause the one or more processors to perform one or more of the processes. Reference to FIGS. 3A and 3B will be made to further illustrate the processes of method 200.

At an optional process 202, a treatment device is positioned in an anatomic lumen at a first location. For example, and with reference to FIG. 3A which provides a detailed view of the region 113 of the lung 104, a treatment device in the form of medical instrument 100 may be positioned in the lumen 122 of anatomic passageway 102. The medical instrument 100 may include a flexible outer catheter 150 through which an inner catheter 152 may extend. In some embodiments the outer catheter may have a working channel diameter of approximately 2 mm and the inner catheter may have an outer diameter of less than approximately 2 mm. In some embodiments, all or a portion of the length of the inner catheter may be surrounded by an insulation jacket to reduce heat loss from a heated liquid flowing through the catheter. A distal portion 154 of the inner catheter 152 includes a shaft 160 which carries an occlusion device 156 and which houses a heating device 158. A distal portion 154 may include a distal opening 164 through which a lumen of the shaft 160 may be in fluid communication with the lumen 122. Optionally, the inner catheter 152 may also carry sensors 166, such as pressure sensors for measuring a pressure within the inner catheter and/or temperature sensors for measuring temperature of a liquid 162 within the inner catheter. Optionally, the inner catheter may also carry mechanical devices 168 such as an auger or other tools to advance or propel the liquid 162, pumps, and/or release mechanisms for releasing the liquid 162 through the distal opening 164. The positioning of the flexible outer catheter 150 and/or the inner catheter 152, including insertion motion, retraction motion, and steering control may be performed with a robot-assisted medical system or may be performed manually by a clinician.

At an optional process 204, factors may be assessed related to the procedure to be performed with the medical instrument 100. For example, either before (e.g., pre-operatively) or after (e.g., intra-operatively) the instrument 100 is positioned in the lumen 122, anatomic images of the lung 104, including the region 113 may be obtained. The images may be obtained by imaging systems using technology such as computerized tomography (CT), magnetic resonance imaging (MRI), fluoroscopy, thermography, ultrasound, optical coherence tomography (OCT), thermal imaging, impedance imaging, laser imaging, or nanotube X-ray imaging. The images may provide information about the type, location, volume and density of the diseased tissue (e.g. a lung tumor, emphysema, chronic bronchitis, brochiectasis), lumen size, lumen volume and may be used to determine a suitable treatment type and a location for delivering the treatment.

At an optional process 206, the anatomic lumen may be prepared for delivery of an endoluminal thermal treatment. In some examples, a lavage treatment may be delivered to the anatomic lumen 122 to remove mucous and expose biofilm. In some examples, a suctioning treatment may be applied to the anatomic lumen to remove mucous and liquid. In some examples, a medicinal treatment may be delivered to the lumen 122.

At a process 208, the occlusion device 156 may be deployed. In some examples, the occlusion device 156 may be an expandable device such as an inflatable balloon, an expandable membrane, or an expandable hood that extends circumferentially around the catheter shaft 160. The occlusion device 156 may have a collapsed configuration as shown in FIG. 3A which allows the distal portion 154 of the inner catheter 152 to be inserted or retracted within the passageway 102. The occlusion device 156 may have a deployed configuration as shown in FIG. 3B in which the occlusion device extends into contact with the wall of the passageway 102 to form a seal or barrier preventing fluid flow proximally of the occlusion device. In some embodiments, the deployed configuration of the occlusion device 156 may further function to lodge the inner catheter 152 in the passageway 102, prevent translation of the inner catheter relative to the passageway. In some embodiments, the occlusion device may expand to a predetermined size and configuration. In some embodiments, the extent of the expansion of the expandable device may be controlled based, for example, on the diameter of the passageway. In some embodiments, the location and extend of the expansion may be monitored by a visualization system. For example, a visualization system may include a fiber optic cable coupled to the catheter and a distal lens to visualize the placement and expansion of the occlusion device. The outer surface of the catheter may include a groove sized to receive the fiber optic cable. In some embodiments, the fiber optic cable is housed within a visualization catheter that fits within the groove. In some embodiments, the extent of the expansion may be monitored and/or controlled by a robot-assisted medical system control system (e.g. control system 1012). For example, the robot-assisted medical system may receive sensor data from the visualization system or from an external imaging system.

At a process 210, a non-compressible fluid is heated in or near the distal portion of the catheter. For example, the heating device 158 within the inner catheter 152 may heat a non-compressible fluid such as liquid 162 to a temperature of less than 100 degrees Celsius. The heating device 158 may, for example, heat the liquid 162 using a radiofrequency (RF) heating element, a resistive heating element, a microwave heating element, an ultrasonic heating element, a magnetic heating element, or a light/laser-based heating element. The liquid 162 may be delivered to the catheter from a liquid source 161 such as a fluid reservoir or another coupled liquid source coupled to a proximal portion of the inner catheter 152. In some embodiments, the catheter may be considered the liquid source. The liquid 162 may be, for example, water (including sterile or de-ionized water), saline, gel, solution, glycerin, or oil that maintains a liquid state at temperatures approaching 100 degrees Celsius. Generally the liquid may be held at a temperature lower than the liquid's vaporization or boiling point. Depending on the components of the liquid, it may be heated to a temperature greater than 100 degrees Celsius while maintaining a liquid state. In some examples, the liquid may be heated to a temperature between approximately 50 and 200 degrees Celsius. In some examples, the liquid may be a gel having a viscosity of between approximately 0.5 and 10,000 centipoise. In some examples, the liquid may be a pure solvent such as water, propylene glycol, diglyme, tetrahydrofurfuryl alcohol, N-methyl-2-pyrrolidone, or solketal. In some examples, the liquid may be comprised of inert or generally inert substances and may comprise a variety of solvents and solutes such as antimicrobial, antibiotic, antifungal, and or aseptic additives to facilitate healing, injury, or other responses. The liquid may further include radiopaque additives such as calcium carbonate, gold flakes, iodine, barium-sulphate, gadolinium or other radiopaque materials that facilitate delivery and monitoring of the delivered liquid. The liquid may include additives that adjust the heat capacity, heat transfer properties, viscosity, and/or osmolarity of the liquid, including starches, polyethylene, or fiber. In some embodiments, the liquid may be bioabsorbable over a period of time such as approximately one to six weeks. In some embodiments, the heating device 158 may also include a pressurization system for pressurizing the liquid 162. The heating device 158 may pressurize the liquid using, for example, a linear actuator, a screw pump, a piston pump, a rotary pump, a diaphragm pump, or a peristaltic pump. In some embodiments, the liquid may be heated by a heating and pressurization device at a proximal portion of the medical instrument or in a reservoir coupled to the medical instrument. The heated liquid may be pressurized and delivered into the proximal portion of the inner catheter and may flow through the inner catheter to the distal portion of the inner catheter before being released from the distal opening. In some embodiments, the liquid may be heated by a heating device coupled to and located externally of the distal opening. In some embodiments, the liquid may serve as a resistor in a resistive circuit. In some embodiments, the liquid may be heated with an exothermic reaction, such as a reaction of zeolite with water.

At an optional process 212, parameters associated with the liquid may be adjusted based on the intended outcome of the treatment. For example, parameters 220 such as the temperature of the liquid 222, the rate of liquid release 224, the volume of released liquid 226, the duration of the liquid release 228, and/or the pressure at which the liquid is released 230 may be adjusted based on the type of disease to be treated, the size of the lumens, the proximity of other passageways, or other environmental factors.

At a process 214, the heated liquid is released from the distal portion of the catheter into the anatomic lumen. For example, the heated liquid 162 may be released from the distal opening 164 into the lumen 122 distally as shown in FIG. 3B. The release of the diffuse liquid 162 may be subject to the adjusted parameters 220. The heated liquid 162 may ablate the bronchial wall 124 and other tissue surrounding the lumen 122, distal of the occlusion device 156. In some embodiments, the heated liquid may be delivered to tertiary bronchi at a rate between approximately 0.1 milliliters per second and 10 milliliters per second. In some embodiments, the delivery of the liquid and the migration of the liquid through the passageway(s) 102 may be monitored with an intra-operative imaging system. For example, the liquid may include radiopaque particles that are visible on intra-operative CT images so that the volume of delivered liquid, duration of the liquid release, rate of the liquid release, and/or the migration of the liquid may be observed and monitored. In some embodiments, the liquid released from the catheter may be dispensed to an expandable member such as an inflatable balloon. The expandable member may contain the migration of the liquid based on the size and shape of an expanded configuration of the expandable member. Various embodiments of expandable members, including balloons, that may receive the dispensed liquid are described in PCT Application (Docket No. P02302-WO) filed Jul. 7, 2020, titled “Systems and Method for Localized Endoluminal Thermal Liquid Treatment,” which is incorporated by reference herein in its entirety.

At an optional process 216, the catheter may be moved as the heated liquid is released from the distal portion of the catheter. For example, the inner catheter 152 may be retracted as the liquid 162 is released from the opening 164. If the occlusion device is mounted to the inner catheter as shown in FIG. 3B, a slippery interface between the bronchial wall and the occlusion device may allow the occlusion device to move while inflated. The diameter of the occlusion device may be increased as the occlusion device is moved proximally into larger diameter passageways. In alternative embodiments, the occlusion device may be coupled to the outer catheter. In the alterative embodiments, the inner catheter may be retracted relative to the outer catheter while the occlusion device remains parked or the occlusion device on the outer catheter may be retracted with the inner catheter and the occlusion device may move while inflated. Alternatively, the inner catheter 152 and/or the outer catheter 150 may be advanced as the liquid 162 is released from the opening 164. Optionally, the occlusion device may be collapsed or partially collapsed while the catheter is moved. In some embodiments, the catheter may be moved with the occlusion device in a fully deployed configuration.

At an optional process 218, the liquid may be removed from the anatomic lumen. For example, suction may be applied through the catheter 152 or through another suctioning instrument to remove at least some of the liquid 162 from the lumen 122.

In some embodiments the method 200 may be used for the treatment of emphysema to induce airway occlusion and lung volume reduction of lung regions distal of the tertiary bronchi 112, such as sixth to tenth generation airways. Emphysema may result in heterogeneously distributed hyperinflation of the alveoli. These hyperinflated segments of the lung may have poor gas exchange and may encroach upon healthier regions of alveoli. Reducing these poorly functioning hyperinflated segments of lung may improve lung function and quality of life. Heating the bronchial passageways, and in some treatments the adjacent arteries, with the heated liquid may induce cell injury and subsequent tissue proliferation and thereby airway and vasculature occlusion. The heat may ablate the bronchial wall, resulting in collagen shrinkage and neo-intima hyperplasia. This may lead to occlusion of the bronchial passageway and subsequent lung volume reduction of that hyperinflated segment. A reduction of air and blood flow to emphysematous tertiary and more distal segments of the lung may redirect air and blood flow from poorly functioning distal segments to better functioning segments. This may improve pulmonary function and quality of life for emphysema patients.

Because of the smaller size of bronchi and the close proximity and small size of the adjacent vasculature, delivery of the heated liquid at the tertiary or sub-tertiary bronchi provides sufficient heat capacity, heat transfer and heat dissipation that primarily results in bronchial wall ablation and, in some cases, airway occlusion and secondarily results in occlusion of the vasculature structures as the healing process proceeds. Additionally, ablating at the tertiary bronchi level may maintain the mucociliary transport, which begins proximal to the tertiary bronchi. Other thermal methods ablate at the bronchus level thereby thermally ablating critical mucociliary transport that can negatively affect the healing process. In some embodiments, a viscous liquid, such a gel, may enable the heat energy to remain in place regardless of gravitational forces during heat transfer from the airways to the surrounding tissue structures including the parenchyma and vasculature. In some embodiments, surface tension in the narrow bronchi may be sufficient to hold a non-viscous liquid, such as a saline solution, in place regardless of the gravitational forces.

For emphysema an ablation through the artery wall to the adventitia but not ablating the bronchial artery may be effective. Treatment parameters including liquid temperature, liquid volume, liquid delivery speed and distance to static position of the heated liquid may be determined and controlled in order to achieve ablation at the effective depth. The treatment parameters, including temperature and depth of ablation of the passageway may be selected based on bronchial wall thickness. The bronchial wall depth may vary in thickness from approximately 0.3 mm to 1.0 mm and may be determined from imaging data, such as intra-operative or pre-operative CT data. Once the wall thickness is determined, the parameters for ablation through the bronchial wall but not through the bronchial arteries may be determined. Using the described endoluminal thermal liquid treatment methods, multiple airways may be simultaneously ablated due to the liquid's ability to traverse into a multitude of airways down to the terminal bronchioles. These treatment methods may be faster as compared to techniques that reduce lung volume with multiple focal treatments. Using the described endoluminal thermal liquid treatment methods, multiple airways may be subsequently ablated from distal to proximal in order to maximize the temperature of each airway treated.

The use of the described systems and methods for treatment of emphysema may be effective in, for example, the third to fifth generation bronchi and may be used in approximately 3 to 6 segments during a procedure. For treatment of emphysema, a temperature of the heated liquid (with a boiling point of about 100 degrees Celsius, such as water, for example) may be just below 100 degrees Celsius or approximately 85 to 99 degrees Celsius at release. For treatment of some disease states, which may include emphysema, liquids at temperatures greater than 100 degrees Celsius (while maintaining a liquid state) may be suitable. Generally, the depth of ablation may extend only through to the adventitia. Therefore, in some embodiments, no thermal damage or subsequent permanent occlusion may occur to the arteries.

In some embodiments, the method 200 may be used for the treatment of chronic bronchitis. Chronic bronchitis may result in hypersecretion of goblet cells which reside within the airway epithelium resulting in excess mucus, frequent infective exacerbations, and chronic productive cough. Ablating these hypersecreting goblet cells while preserving the intercellular matrix may cause regeneration of more normally secreting goblet cells and consequentially, a reduction of mucus production, infective exacerbations and cough. When treating chronic bronchitis, the depth and uniformity of epithelium ablation may be significant factors. If the thermal energy delivery is too shallow, the goblet cells may be insufficiently ablated. If the thermal energy delivery is too deep, the lamina propria, smooth muscle cells, and submucosa may be ablated, causing neomucosal hyperplasia and potentially resulting in unwanted airway obstruction. Depth of ablation may be controlled by the temperature and heat capacity of the liquid to target goblet cells at a depth of 0.05 mm to 0.15 mm within the airway epithelium that lines the bronchial lumen. Goblet cells reside in the epithelium until the terminal bronchi, and the heated liquid delivery allows for ablation of the airway to the terminal bronchi for complete goblet cell ablation. Ablating the cilia and hypersecreting goblet cells while preserving the cellular matrix may allow for the regrowth of healthier goblet cells and cilia without airway obstruction. The healthy regrowth may reduce mucus production, improve lung function, and improve quality of life.

Chronic bronchitis airways may form pleats. Using any of the endoluminal thermal liquid treatments described herein, the heated liquid may fill the pleats to ablate otherwise inaccessible goblet cells and cilia. This treatment may improve efficiency compared to devices with focal treatments that may be repeated multiple times to achieve the same treatment results. (see heated balloon application for clinical details). For chronic bronchitis an ablation depth of 0.05 mm to 0.15 mm may be effective. Treatment parameters including liquid temperature, liquid volume, liquid delivery speed and distance to static position of the heated liquid may be determined and controlled in order to achieve ablation at the effective depth. The use of the disclosed systems and methods for treatment of chronic bronchitis may be effective in, for example, the third to fifth generation bronchi and may be used in any or all of the lobes of the lung. For treatment of chronic bronchitis, a temperature of the heated liquid (with a boiling point of about 100 degrees Celsius, such as water, for example) may be approximately 70 to 95 degrees Celsius at release. For treatment of some disease states, liquids at temperatures greater than 100 degrees Celsius (while maintaining a liquid state) may be suitable.

The lumen may be generally tapered and somewhat cylindrical, with ridges, bumps and other imperfections. The heated liquid provides for a controlled ablation depth. For example, the temperature of the liquid may be adjusted to provide a low rate of ablation that allows for highly controlled ablation depth. As compared to rigid ablation devices that may force the lumen to comply with the ablation device creating areas of higher-pressure contact and compression of the epithelium which can result in uneven depth of ablation, the use of a heated liquid for ablation may provide for complete ablation of the irregular surface of the bronchial lumen as the liquid conforms to the shape of the lumen. Applying the heated liquid to the airway may also provide for safe and consistent overlap of ablation zones since it relies simply on heat transfer through a tissue. Other energy devices that rely on the electrical properties of the tissue for ablation can result in varying ablation depths based on whether the tissue has been previously ablated. Applying heated liquid to the airway also allows for ablation of bifurcated bronchial passageways at bronchial carinas by delivering a volume of liquid that is slightly greater than the targeted airway lumen being filled. This slight overfilling results in a truncated Y-shaped ablation pattern.

In some embodiments, the method 200 may be used for the treatment of bronchiectasis. Bronchiectasis is an abnormal, chronic enlargement of the bronchi that may occur due to infection or noninfectious factors. Bronchiectasis may result in enlarged bronchial pockets and damaged cilia. A decreased ability to clear secretions due to damage of the cilia may accompany the enlargement of the bronchi. Failure to clear secretions allows microbes to collect in them along with the formation of biofilms, which leads to more secretions and inflammation that further damage the airways, causing still further dilation. Bronchiectasis maybe localized, occurring in a single portion of the lung or may be diffuse, occurring throughout the lungs. It is the major lung abnormality of cystic fibrosis, causing infective exacerbations and pneumonia. Applying heated liquid to the airway may improve bronchiectasis in one or more ways. For example, applying the heated liquid to the airway may kill biofilm, bacteria, fungus and other microbes. Ablating the damaged cilia with heated liquid may result in a regrowth of more normal cilia. Cilia reside in the epithelium until the terminal bronchi.

Ablating the submucosa in the enlarged portions of the airway may result in neomucosal hyperplasia which reduces the size of the pockets and facilitates mucus clearance and reduced nidus for infection. Reducing the biofilm, bacteria, fungus and other microbes and improving the mucociliary transport, along with reducing the pockets, results in fewer infective exacerbations may improve the quality of life for the patient and may reduce hospitalizations and associated costs. The suitable temperature of the liquid and the duration for which it is applied to the cilia and submucosa may be determined, monitored and controlled. In some embodiments, the liquid, such as a viscous gel, may conform to the shape of the lumen to provide complete ablation of the pocket as well as irregular surface of the lumen. Applying the heated liquid to the airway may also provide for safe and consistent overlap of ablation zones since it relies simply on heat transfer through a tissue. Other energy devices that rely on the electrical properties of the tissue for ablation can result in varying ablation depths based on whether the tissue has been previously ablated.

For bronchiectasis an ablation through the artery wall to the adventitia but not ablating the bronchial artery may be effective. Treatment parameters including liquid temperature, liquid volume, liquid delivery speed and distance to static position of the heated liquid may be determined and controlled in order to achieve ablation at the effective depth. The parameters, including temperature and depth of ablation may be selected based on bronchial wall thickness. The bronchial wall depth may vary in thickness from approximately 0.3 mm to 1.0 mm and may be determined from imaging data, such as intra-operative or pre-operative CT data. Once the wall thickness is determined, the time to ablate through the bronchial wall but not through the bronchial arteries may be determined. The use of the disclosed systems and methods for treatment of bronchiectasis may be effective in, for example, the third to fifth generation bronchi and may be used in any of three lobes of the lung during a procedure. For treatment of bronchiectasis, a temperature of the heated liquid (with a boiling point of about 100 degrees Celsius, such as water, for example) may be just below 100 degrees Celsius or approximately 85 to 99 degrees Celsius during treatment. For treatment of some disease states, which may include bronchiectasis, liquids at temperatures greater than 100 degrees Celsius (while maintaining a liquid state) may be suitable.

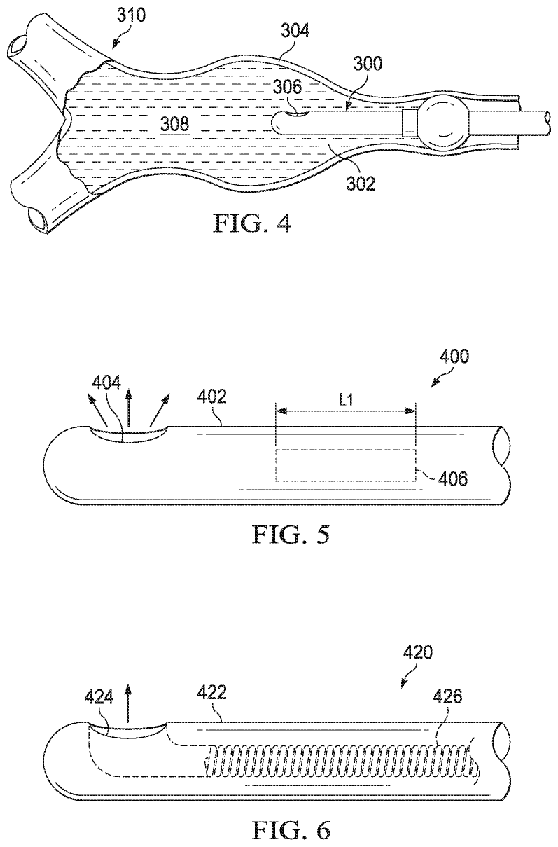

When treating bronchiectasis using the method 200, a vigorous lavage or scouring may be used at process 206 to remove mucus and expose biofilm. For the process 202, the catheter may be positioned such that the distal opening is at the midway point of the pocket. As shown in FIG. 4, a catheter 300 (e.g. the inner catheter 152) may be positioned within the lumen 302 of a bronchial pocket 304 such that a distal opening 306 of the catheter 300 is positioned at approximately the beginning of the pocket 304. At the process 204, the dimensions and volume of the pocket 304 may be determined from pre-operative or intra-operative images to determine a volume of liquid 308 sufficient to ablate the walls of the pocket. Alternatively or additionally, at process 214, the heated liquid may have radiopaque properties and may be visible on intra-operative imaging such that the volume of dispensed liquid 308 may be monitored until a truncated Y formation 310 is observed in the images. At the processes 212 and 214, the time duration parameter may be selected to allow the liquid to ablate the epithelium. In some examples, a duration of approximately 10 to 60 seconds may be suitable. At the process 218, some or all of the liquid may be removed with a suction catheter to restore airflow through the lumen 302 to the distal lung regions. The process for treating bronchiectasis may be repeated for multiple afflicted airways, starting generally distally and proceeding proximally.

In some embodiments, the method 200 may be used for the treatment of a lung tumor. In some embodiments, the lung tumor may have a diameter of approximately 2 cm or less. Delivery of heated liquid to the distal bronchi may provide conduction and migration of thermal energy to the entire sub-segmental microvasculature and parenchyma resulting in the ablation of the tumor and tumor margin via cellular death and ischemia. Subsequent to ablation, naturally occurring macrophage removal of the ablated segmental tissue may result in removal of the ablated tissue because it is not thermally fixed or altered. Ablated tissue that is not thermally fixed may be subsequently removed by the body. When the ablation follows the anatomical boundaries, the result may be complete or substantially complete removal of the entire anatomical structure that includes the tumor, with results similar to a pulmonary segmentectomy. The ablation of a margin of a tumor may result in a lower local recurrence rate. This method of treatment may also promote an immunostimulation effect. In traditional ablation methods that directly provide thermal ablation via radiofrequency, high intensity focused ultrasound, microwave, or radiotherapy, tumor antigens and epitopes are significantly denatured and destroyed, reducing the ability for the immune system to identify the diseased cells. Using a heated liquid, as in method 200, may kill the tumor cells while preserving the cellular proteins and antigens, allowing the immune system to recognize the diseased cell type which enables the immune system to attack tumor cells elsewhere in the body. This is demonstrated by the abscopal effect, which can occur following certain tumor ablations.

With the method 200, heat can be projected from the catheter via the flow of the liquid. The liquid may flow from the catheter placed proximally to the tumor and may follow the natural airways in the lung. This allows for a simpler catheter placement because there is no need to place the catheter within the tumor or to create new channels in the lung. Additionally, because the liquid may flow to and around the tumor, via the airway lumens, there is minimal risk of pneumothorax from a catheter placed too closely to the lung surface. Thus, tumors at the lung surface may be evenly heated.

FIG. 5 illustrates a distal portion of a catheter 400 (e.g. the catheter 152) that may be used to perform the method 200. The catheter 400 may include a shaft 402 with side-facing distal opening 404. In this embodiment, a heating device 406 may generate heat, for example, via resistive wire heating or through radiofrequency, microwave, or high intensity focused ultrasound. In this embodiment, the heating device 406 may be fixed in a stationary position relative to the shaft 402 and may have a length L1 of approximately 1 mm to 10 mm.

FIG. 6 illustrates a distal portion of a catheter 420 (e.g. the catheter 152) that may be used to perform the method 200. The catheter 420 may include a shaft 422 with side-facing distal opening 424. In this embodiment, a heating device 426 may generate heat, for example, via an RF or resistive heating coil that extends along the entire or a substantial length of the catheter 420. In this embodiment, the liquid may be heated by the coil 426 as it is conveyed from a proximal portion of the catheter 420 to the distal portion of the catheter.

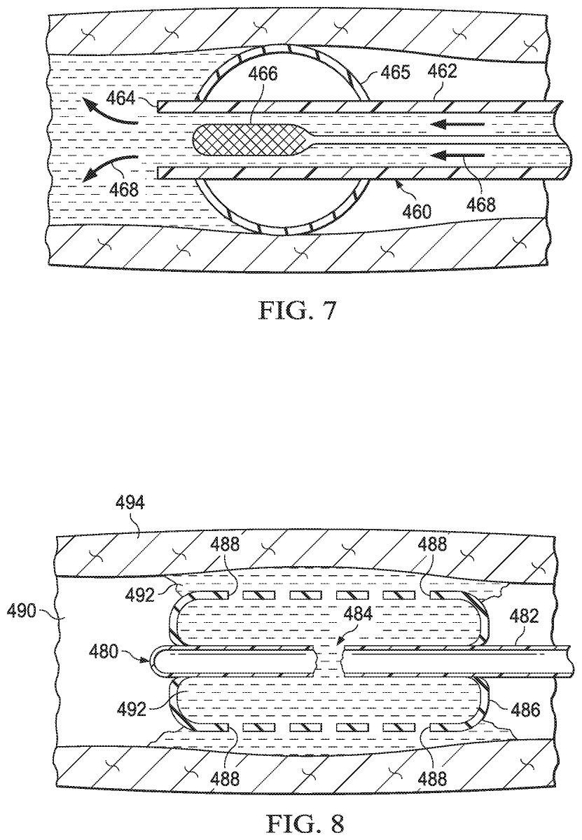

FIG. 7 illustrates a distal portion of a catheter 460 (e.g. the catheter 152) that may be used to perform the method 200. The catheter 460 may include a shaft 462 with a distally-facing distal opening 464 and an occlusion device 465 coupled to the shaft. In this embodiment, a heating device 466 may generate heat, for example, via a radiofrequency heating wire mesh that extends within the shaft 462. In this embodiment, the liquid 468 may be heated by the wire mesh heating device 466 while it is contained within the distal portion of the catheter 460.

FIG. 8 illustrates a distal portion of a catheter 480 (e.g. the catheter 152) that may be used to perform the method 200. The catheter 480 may include a shaft 482 with one or more side-facing distal openings 484 and an expandable member 486 coupled to the shaft. In some embodiments, the expandable member 486 may be a permeable sack having a plurality of micro holes 488. In some embodiments, the expandable member 486 may be woven and/or may have a cheese cloth like permeability. The expandable member 486 may have an expanded diameter that may be approximately the diameter of the lumen 490. The expandable member 486 may be expanded by the heated liquid 492 and into contact with the bronchial walls 494 surrounding the lumen 490. Once the expandable member 486 is turgid, the liquid 492 may flow through the micro holes 488 to heat the bronchial walls 494 surrounding the lumen 490.

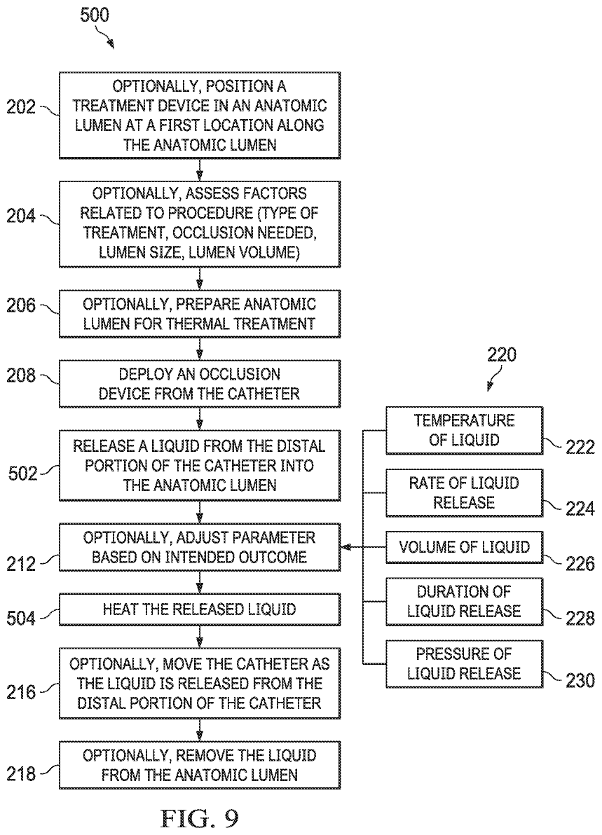

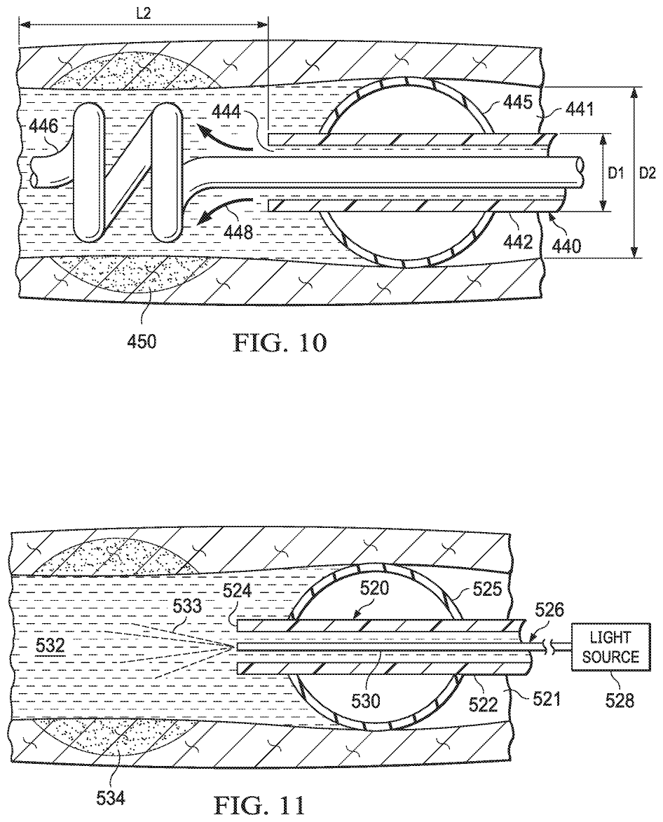

FIG. 9 is a flowchart illustrating a method 500 for applying a thermal energy treatment to an endoluminal passageway. The method 500 may include processes in common with method 200 as indicated by the same numerical identifier. Processes unique to method 500 are as described. In this embodiment, a liquid may be heated after being released from the distal portion of the catheter. At a process 502, liquid may be released from the distal portion of the catheter into the anatomic lumen. The release of the liquid may be subject to the adjusted parameters 220. In some embodiments, the delivery of the liquid and the migration of the liquid through the passageway(s) 102 may be monitored with an intra-operative imaging system. For example, the liquid may include radiopaque particles that are visible on intra-operative x ray or CT images so that the volume of delivered liquid, duration of the liquid release, rate of the liquid release, and/or the migration of the liquid may be observed and monitored. At a process 504, the released liquid may be heated externally of the catheter using, for example a radiofrequency heating device, a light or laser-based heating device, a resistive heating device, an ultrasonic heating device, a magnetic heating device or a microwave heating device. The liquid heated externally of the catheter may ablate the airway tissue and/or the adjacent vasculature as described in any of the embodiments above. In some embodiments, the liquid may be pre-heated by a heating device as previously described in method 200 and may be further heated externally of the catheter as described in method 500. The method 500 is further described with respect to FIGS. 10 and 11.

FIG. 10 illustrates a distal portion of a catheter 440 that may be used to perform the method 500 within a lumen 441. The catheter 440 may include a shaft 442 with distally-facing distal opening 444. An occlusion device 445 is coupled to the shaft 442. In this embodiment, a heating device 446 may extend distally of the distal portion of the catheter 440. The heating device 446 may be a radiofrequency coil formed of a wire that may have a diameter of approximately 1 mm, for example. Liquid 448 flowing from the distal opening 444 may be heated by the heated coil distally of the distal opening 444, within the anatomic lumen 441. The heated liquid 448 may induce formation of an ablation zone 450 in the bronchial wall surrounding the lumen 441. In some embodiments, the catheter 440 may have an outer diameter D1 of approximately 2 mm and may extend within the anatomic lumen 441 having an inner diameter D2 of approximately 4 mm. In some embodiments the heating device 446 may have a coil length L2 of approximately 8 mm.

FIG. 11 illustrates a distal portion of a catheter 520 that may be used to perform a method 500 within a lumen 521. The catheter 520 may include a shaft 522 with distally-facing distal opening 524. An occlusion device 525 is coupled to the shaft 522. In this embodiment, a heating device 526 may extend within the catheter 520. The heating device 526 may be a light source 528, such as a laser, coupled to an optical transmission member 530, such as one or a plurality of fiber optic cables. The optical transmission member 530 may convey light from the light source 528 to the distal portion of the catheter 520. In some embodiments, the optical transmission member may extend through the distal opening 524, but in some embodiments, the optical transmission member may terminate proximal of the distal opening 524. In some embodiments, for example if the optical transmission member includes a plurality of optical fibers, the optical transmission member may terminate both distally and proximally of the distal opening 524. Liquid 532 flowing from the distal opening 524 may be heated by light 533 from the optical transmission member 530 distally of the distal opening 524, within the anatomic lumen 521. The heated liquid 532 may induce formation of an ablation zone 534 in the bronchial wall surrounding the lumen 521.

In some embodiments the liquid 532 may include an additive, such as a dye, to increase the light absorption capacity of the liquid. In some embodiments, the optical transmission member 530 may include lenses, mirrors, or other optical components at the ends of optical fibers to enhance dispersal of the optical energy within the liquid. In some embodiments, the light source 528 may include a pulsed laser. In some embodiments, a temperature sensor may extend into or otherwise contact the liquid 532. Temperature data for the liquid obtained from the temperature sensor may be used to modulate power to the light source 528 to control heating of the liquid to a specific temperature. In some embodiments, an opacity differential within the released liquid may improve the transmission of the thermal energy. With the liquid disposed in a cylindrical airway, the liquid 532 at the exterior (e.g., closest to the bronchial wall) may be relatively opaque to the light 533 and the liquid 532 at the interior (e.g., near the center of the lumen 521) may be relatively translucent to the light 533. The translucent interior may thus for a liquid tube through which the light 533 may be conducted, effectively allowing the liquid tube to serve as a continuation of the optical transmission member 530. The opacity of the liquid may be achieved using various additives, such as electrically charged particles that migrate to the edges of the liquid to create opacity.

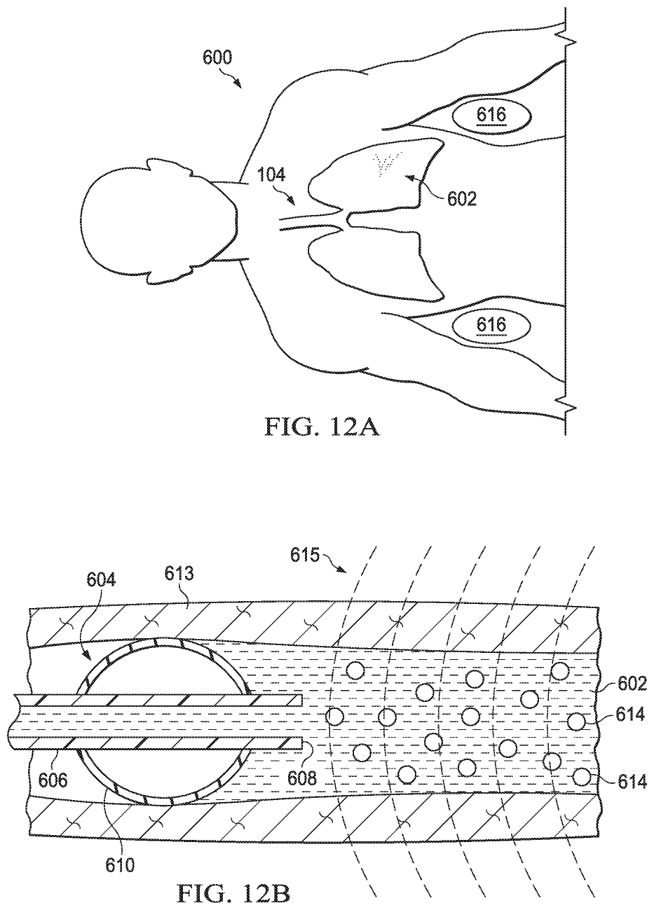

FIG. 12A illustrates a portion of a patient anatomy 600 that may include the anatomic structure 104 into which a liquid 602 may be dispensed by a catheter 604, as shown in FIG. 12B. FIG. 12B illustrates a distal portion of the catheter 604 (e.g. the catheter 152). The catheter 604 may include a shaft 606 with a distally-facing distal opening 608 and an occlusion device 610 coupled to the shaft. In this embodiment, the liquid 602 dispensed from the catheter 604 may include magnetic particles 614. The liquid 602 with magnetic particles 614 may be exposed to an oscillating magnetic field 615 from a magnetic field generator 616 which may be, for example, positioned on opposite sides of the anatomic structure 104. The resistance to motion of the excited magnetic particles 614 may be translated to resistive heating of the liquid 602. As described in previous embodiments, the heated liquid may cause ablation of the bronchial wall 613. In some embodiments, the liquid 602 may be washed or suctions from the lumen to remove the magnetic particles after the ablation therapy. In some embodiments, a temperature sensor may extend into or otherwise contact the liquid 602. Temperature data for the liquid obtained from the temperature sensor may be used to modulate power or frequency of the magnetic field generator 616 to control heating of the liquid to a specific temperature.

In an alternative embodiment, the liquid may be conductive or comprise conductive particles, such as iron oxide. When the conductive liquid is exposed to the oscillating magnetic field 615, eddy currents may be induced in the liquid which causes the liquid to heat by induction heating. In some embodiments, a conductive object, such as a wire or coil may extend from the catheter into the liquid. The conductive object, when exposed to the oscillating magnetic field 615 may induce eddy currents and heat the conductive object through induction. The heat from the conductive object may be conducted to the liquid. In some embodiments, a temperature sensor may extend into or otherwise contact the liquid. Temperature data for the liquid obtained from the temperature sensor may be used to modulate power or frequency of the magnetic field generator to control the inductive heating of the liquid or the conductive object to a specific temperature.

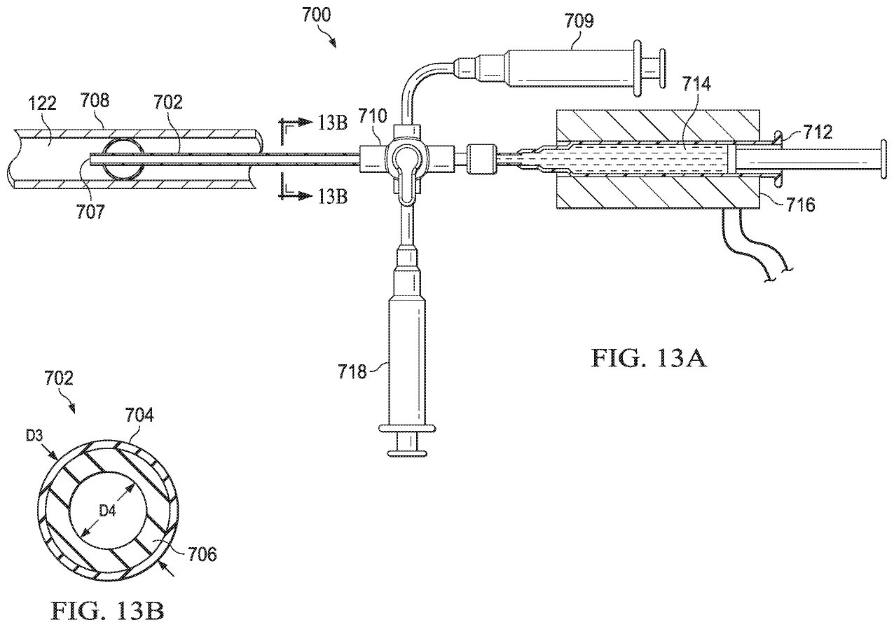

FIG. 13A illustrates a medical instrument system 700 including a catheter 702. The catheter 702 may be similar to inner catheter 152 and may, in some embodiments, be inserted through an outer catheter such as outer catheter 150 which may be manually or robotically actuated. FIG. 13B illustrates a cross-sectional view of the catheter 702 which includes an outer tube 704 and an inner tube 706. In some embodiments, the outer tube 704 may be formed of an elastomeric material such as PEBEX. In some embodiments, the outer tube 704 may have an outer diameter D3 of approximately 0.070 inches and a wall thickness of approximately 0.003 inches. In some embodiments, the inner tube 706 may be formed of a thermal insulation material such as silicone. In some embodiments the inner tube 706 may have an inner diameter D4 of approximately 0.030 inches and a wall thickness of approximately 0.020 inches. A distal portion of the catheter 702 may include a distal opening 707 through which the catheter 702 may be in fluid communication with a lumen (e.g. lumen 122) of a patient.

An occlusion device 708 (e.g. the occlusion device 156) is coupled to the catheter 702. The occlusion device 708 may be an inflatable device such as a silicone balloon. The occlusion device 708 may be in fluid communication with an inflation device 709 via the catheter 702. In some embodiments the inflation device 709 may be a syringe including a fluid reservoir for containing a predetermined amount of inflation medium that may be injected into the occlusion device 708 to inflate the occlusion device. In some embodiments, for example, a 1 cm balloon occlusion device may be inflated with 1 cc of air from the syringe inflation device. The inflation device 709 may be coupled to a proximal portion of the catheter 702 via a valve 710, such as a stopcock.

A proximal portion of the catheter 702 may also be coupled via the valve 710 to a liquid source such as a fluid reservoir 712 which contains a non-compressible fluid 714, such as a liquid. The liquid 714 in the reservoir 712 may be heated by a heating device 716. The liquid 714 may be, for example, water, saline, gel, glycerin, solution, or oil that maintains a liquid state at temperatures approaching 100 degrees Celsius. Depending on the components of the liquid, it may be heated to a temperature greater than 100 degrees Celsius while maintaining a liquid state. Glycerin and oil-based liquids may, for example, have boiling points greater than 100 degrees Celsius and thus may be used in endoluminal thermal liquid treatment at temperatures higher than 100 degrees Celsius. In some examples, the liquid may be heated to a temperature between approximately 50 and 200 degrees Celsius. The liquid 714 may include any of the liquid materials or additives described in other embodiments. In one embodiment, the reservoir 712 may be a syringe and may contain 3 cc of liquid that may be heated to approximately 98 degrees Celsius by the heating device 716.

The proximal portion of the catheter 702 may also be coupled via the valve 710 to a flush reservoir 718 which contains a flushing medium such as air or another fluid that may be used to flush the catheter 702. In some embodiments, the flush reservoir 718 may be a syringe that contains 1 cc of air for flushing the catheter 702.

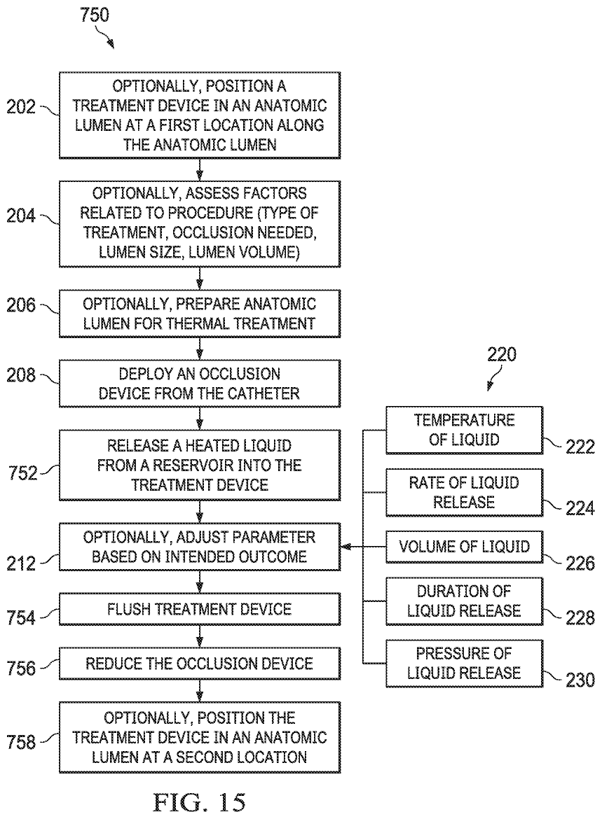

FIG. 15 is a flowchart illustrating a method 750 for applying a thermal energy treatment to an endoluminal passageway. The method 500 may include processes in common with method 200 as indicated by the same numerical identifier. Processes unique to method 750 are as described. In this embodiment, a liquid may be heated after being released from the distal portion of the catheter. Method 750 will be described with reference to medical instrument 700 but may be used with any instrument system in which the liquid is heated proximally of a distal portion of the delivery catheter. At the process 208, the occlusion device 708 may be deployed by inflating the silicone balloon with the air released from the inflation device 709. Releasing the air from the inflation device 709 may include operating the valve 710 to open a through flow channel between the inflation device 709 and the occlusion device 708. In the inflated configuration, the occlusion device 708 occludes the lumen 122, restricting backflow of the heated liquid. At a process 752, the heated liquid 714 may be released from the reservoir 712 into the catheter 702. In some embodiments, a predetermined volume of the liquid 714 may be heated by the heating device 716 to a temperature of approximately 98 degrees Celsius and may be released into the catheter. Releasing the liquid from the reservoir 712 may include operating the valve 710 to open a through flow channel between the reservoir 712 and the catheter 702. In an alternative embodiment, the reservoir may contain more than the predetermined volume of heated liquid, and the predetermined volume may be measured and removed from the reservoir for release into the catheter. At a process 754, the catheter 702 may be flushed of the liquid 714. More specifically, fluid, such as air, from the flush reservoir 718 may be released into the catheter 702 to flush the heated liquid 714 from the catheter. The air flush may also serve to cool the catheter 702. Releasing the fluid from the reservoir 718 may include operating the valve 710 to open a through flow channel between the reservoir 718 and the catheter 702. The heated liquid 714 may ablate the tissue surrounding the lumen 122 as previously described. In some embodiments, a duration of approximately 5 minutes may be sufficient for the heat to transfer to the tissue. At a process 756, after heat has transferred from the heated liquid to the tissue, the occlusion device 708 may be deflated. At an optional process 758, the distal portion of the catheter 702 may be moved to another location within the same bronchi or to a different bronchus for repeat of the method 750 at a second location.

The instrument 700 may minimize heat loss through the catheter because the heated liquid will be in contact with the insulating silicone inner tube 706. The air flush after the injection of the liquid 714 may minimize the temperature of the catheter 702 so that adjacent instrumentation, such as a robotically controlled endoscope is not damaged by transferred heat.

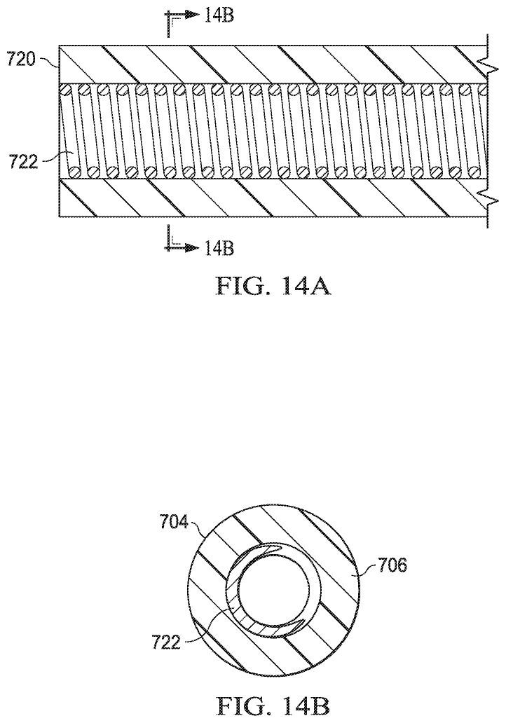

If length of the catheter 702 is too long or if the catheter is not sufficiently insulated, the temperature of the heated liquid 714 may be become sufficiently lowered during delivery down the length of the catheter that the liquid may not perform the ablation as intended. In an alternative embodiment, all or a portion of the catheter may be heated along its length to maintain the temperature of the heated liquid. FIG. 14A illustrates a side cross-sectional view of a catheter 720 that may replace catheter 702 in instrument 700. The catheter 720 includes a heated coil 722 that extends the length or a partial length of the catheter 720. As shown in the cross-sectional view of FIG. 14B, the heated coil 722 may extend around the inner diameter of the inner tube 706. When conducting treatment with the heated catheter 720, liquid released into the catheter 720 may be pre-heated by a heating device 716 or may be released from an unheated reservoir. The volume of liquid released into the catheter 720 may be approximately equal to the volume of the lumen of the catheter. For example, a liquid volume of 0.68 cc may fill the lumen of the catheter 720. The heating coil 722 may be powered either before or after the liquid is released into the catheter, and the heat from the coil may heat the liquid to a predetermined temperature such as 98 degrees Celsius. After the liquid reaches the predetermined temperature the heating coil 722 may be powered off and a predetermined volume of the heated liquid may be released into the patient lumen 122. The air flush of process 754 may have the effect of cooling the catheter 720.

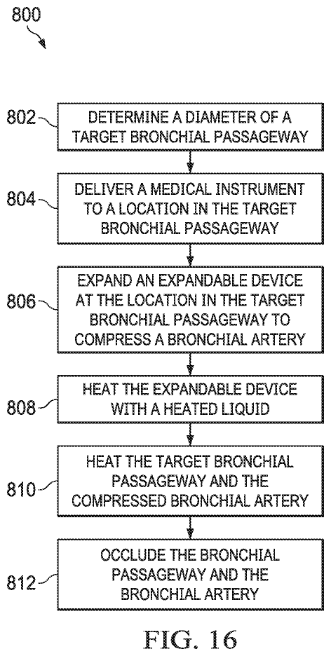

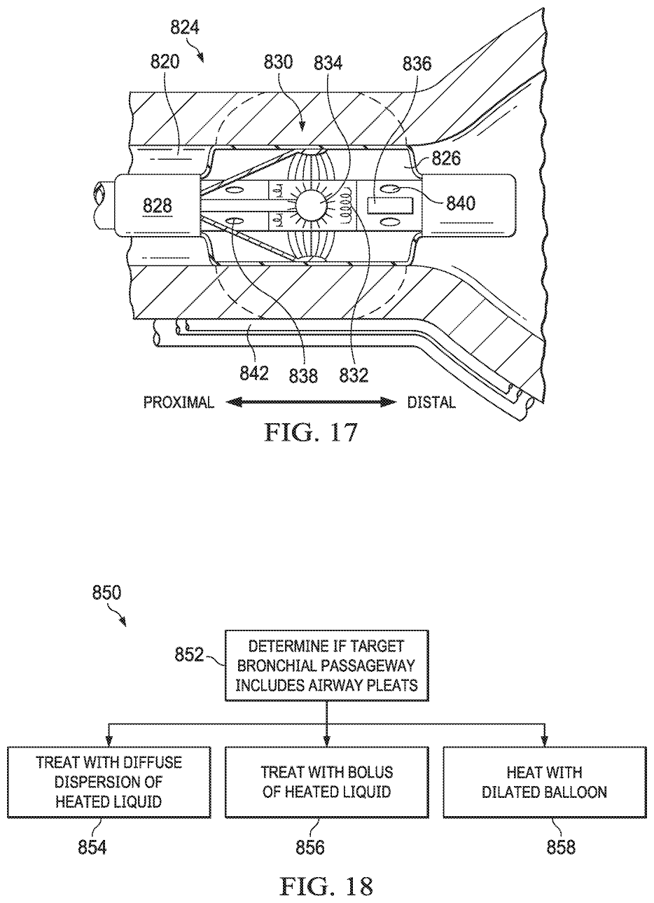

FIG. 16 illustrates a method 800 that may be used to reduce regions of hyperinflated parenchyma that may be associated with conditions such as emphysema. Reduction of the emphysematous parenchyma may be influenced by ablation of the bronchial artery that extends adjacent to the bronchus that leads to the diseased parenchyma. The method 800 is described with further reference to FIG. 17. At a process 802, a size component of a bronchial passageway may be determined. For example, a diameter or a volume of a bronchial passageway 820 may be determined from pre-operative images, intra-operative images, or model estimates.

At a process 804, a medical instrument may be positioned at a location in the target bronchial passageway. For example, as shown in FIG. 11, a medical instrument system 824 may include an expandable device 826 and may be positioned within bronchial passageway 820. The expandable device 826, such as a balloon, may have a generally cylindrical shape and may be coupled to a distal end of a flexible catheter 828. In some embodiments, the balloon may have a length of approximately 5-10 mm and may be semi- or non-compliant. The balloon diameter may be, for example, between 3 and 10 mm. The system 824 may optionally include a heating system 830 that includes bipolar radiofrequency electrodes with an electrode 832 positioned within the expandable device 826, for example on a distal portion of the catheter 828 and with an electrode 834 positioned on a surface of the expandable device. In some embodiments, a plurality of electrodes may be placed within the expandable device and a plurality of electrodes may be placed on the surface of the expandable device. The heating system 830 may also include a temperature sensor 836. Liquid may flow into the expandable device 826 through fluid inlet 838 and may flow out of the expandable device through fluid outlet 840.

At a process 806, the expandable device 826 may be inflated and may compress a bronchial artery 842 extending along the bronchial passageway 820. Compressing the bronchial artery 842 may restrict blood flow through the bronchial artery. By eliminating the heat dissipating blood flow, the following ablation processes may be more effective and efficient. At a process 808, a heated liquid within the expandable device 826 may heat the expandable device. In some embodiments, the liquid is heated within the expandable device 826 by the heating system 830. Additionally or alternatively, the liquid may be heated proximally of the expandable device 826 and may be introduced to expandable device already heated. At a process 810, the heat from the liquid and the expandable device may heat the bronchial passageway 820 and the compressed bronchial artery 842. At a process 812, the heat may cause occlusion of the bronchial passageway and the bronchial artery. For example, the heat may be sufficiently high to cause neointimal hyperplasia of the bronchial artery 842 and occlusion of the bronchial passageway 820. The occlusion may thus result in volume reduction of emphysematous parenchyma distal of the treatment location. Ablating the bronchial artery in addition to the bronchial wall may reduce blood supply to the injured airway wall during the neointimal hyperplasia process, resulting in increased hyperplasia and occlusion.

In some embodiments, the emphysematous parenchyma may be reduced by only ablating the bronchial wall, without ablating and occluding the bronchial artery. The neointimal hyperplasia resulting from the ablation occludes a plurality of airways from the subsegmental level to the terminal bronchioles. This occlusion of airways may result in volume reduction of emphysematous parenchyma. By occluding the airways, and not the bronchial artery, blood supply to the injured airway wall may be maintained during the neointimal hyperplasia process. In some embodiments the method 800 may be used in combination with (e.g., following) a diffuse heated liquid ablation as described in method 200.

FIG. 18 illustrates a method 850 that may be used to treat bronchial airway pleats that may be associated with conditions such as chronic bronchitis. An effective treatment for hypersecreting goblet cells associated with chronic bronchitis may include treatment of the epithelial lining of any airway pleats. At a process 852, intraoperative or preoperative images may be used to determine if a target bronchial passageway includes airway pleats. At a process 854, the surface of the airway pleats may be heated by a diffuse dispersion of heated liquid that may fill the pleat spaces and may displace mucous that may otherwise impede thermal transfer of the heated liquid to the goblet cells within the pleats. For example, a method such as method 200 may be used for diffuse dispersion of heated liquid. At a process 856, which may be performed as an alternative or in addition to processes 854 and 858, a bolus of liquid may be injected into the airway, creating a temporary head pressure that may serve to temporarily expand the pleats and allow the heated liquid to access the goblet cells within the pleats. At a process 858, a heated balloon may be deployed to flatten the pleats and displace mucous that may otherwise impede thermal transfer from the heated balloon to the goblet cells within the pleats.

In some embodiments, reliable lung tumor removal may be achieved by occlusion of the bronchial lumen, the pulmonary artery and the bronchial artery. In some embodiments, a heated balloon or diffuse heated liquid treatment (e.g. method 200) may occlude the bronchial lumen, the pulmonary artery, and the bronchial artery. Sometimes, however, these treatments alone may not be sufficient for full occlusion of all structures. For example, the elastic limit of the bronchial wall may be reached before a heated dilated balloon may cause collapse of the pulmonary artery. An incomplete pulmonary artery collapse may permit continued blood flow and heat dissipation associated with the blood flow. This continued blood flow and heat dissipation may limit the extent of arterial ablation and the subsequent neointimal hyperplasia and may result in a partial infarction or no infarction at all.

In some embodiments, a total acute collapse of the pulmonary artery, via the heated dilated balloon, may not be required to achieve total permanent occlusion of the pulmonary artery. This is because heat to the inner diameter of the artery closest to the heated dilated balloon will cause platelet activation and aggregation which may be sufficient to create acute occlusion. Acute occlusion may create hemostasis which eliminates heat dissipating blood flow through the pulmonary artery, allowing for heat conduction through the static blood and resulting in ablation of the entire circumference of the pulmonary artery. A complete circumferential ablation of the pulmonary artery may maximize the potential for complete permanent neo-intimal hyperplasia occlusion, resulting in a maximum propensity for parenchymal infarction. If, however, platelet activation and aggregation of a partially occluded pulmonary artery from the heated balloon device does not create acute hemostasis, there may be blood flow in the pulmonary artery which could act as a heat dissipater potentially prohibiting ablation of the entire circumference of the pulmonary artery. Failure to ablate the entire circumference of the pulmonary artery may reduce possibility of permanent pulmonary occlusion via neo-intimal hyperplasia.

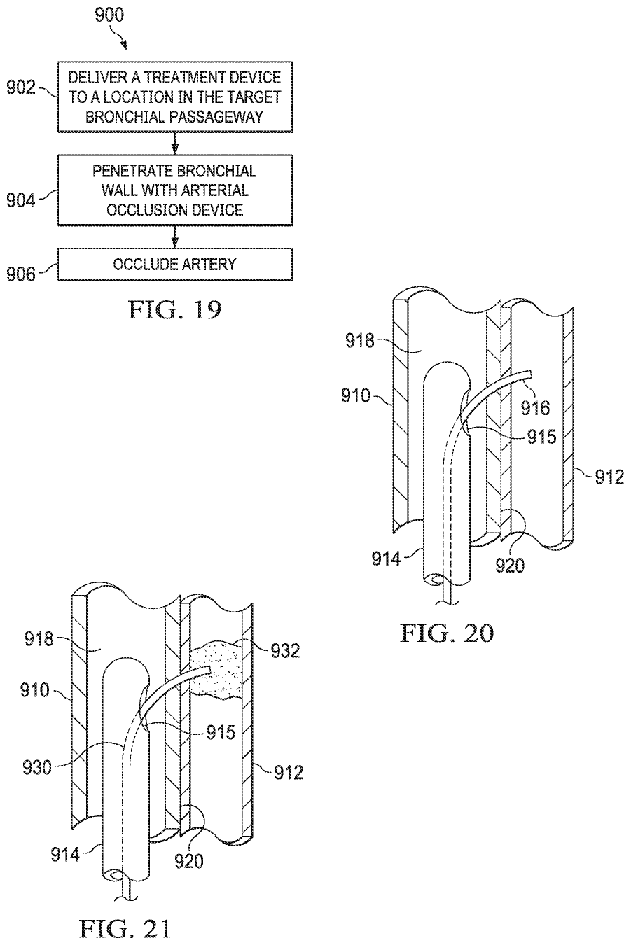

FIG. 19 illustrates a method 900 that may be used to occlude vasculature, including the pulmonary artery that extends along a bronchial passageway to aid in the infarction, necrosis, and subsequent lung tumor removal. At a process 902, a medical instrument may be delivered to a location in a target bronchial passageway. As previously described, a medical instrument may be coupled to a robot-assisted manipulator and navigated to the target location or may be manually positioned at the target location. At a process 904, the medical instrument may penetrate the bronchial wall with an arterial occlusion device. The arterial occlusion device may be moved from the bronchial passageway, through the bronchial wall, and into the artery (e.g., the pulmonary artery) adjacent the bronchial passageway. At a process 906, the arterial occlusion device may occlude the artery by creating an acute clot that prevents blood flow. The stasis of blood may prevent bleeding from the artery into airway and may allow for conduction of heat through the clot providing for complete circumferential ablation of the artery. This may maximize the potential for complete permanent neointimal hyperplasia occlusion. Optionally, the risk of bleeding between puncture site of the airway and the pulmonary artery may be reduced by RF cauterization, an application of fibrin glue, the application of a heated dilated balloon tamponade, the application of an unheated dilated balloon tamponade. Optionally, the other methods such as the diffuse heated liquid ablation method 200 or the localized heated dilation balloon ablation method of incorporated by reference PCT Application (Docket No. P02302-WO) filed Jul. 7, 2020, titled “Systems and Method for Localized Endoluminal Thermal Liquid Treatment,” may be further used to perform ablation of the pulmonary artery, the bronchial artery, and/or the bronchial passageway.

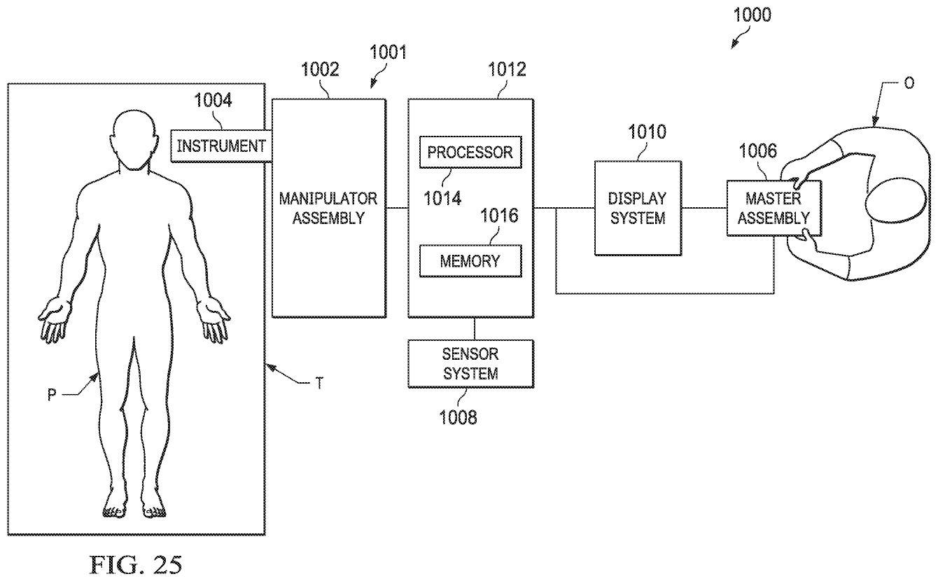

FIGS. 20-25 illustrate examples of arterial occlusion devices that may be used to perform the method 900. FIG. 20 illustrates a bronchus 910 having an adjacent pulmonary artery 912. A catheter 914 may deliver an arterial occlusion device 916 into a lumen 918 of the bronchus 910. The location of the pulmonary artery 912 relative to the bronchus 910 may be determined using, for example, intra-operative imaging. The catheter 914 may be rotated to the direction of the pulmonary artery 912 and the arterial occlusion device 916 may be advanced through a distal opening 915 in a side of the catheter, though a bronchial wall 920, and into the pulmonary artery 912. In this embodiment, the arterial occlusion device may be an RF wire. After placement in the pulmonary artery 912, the RF wire may be activated to cause platelet aggregation and the formation of an occlusive clot. With blood flow in the pulmonary artery 912 blocked by the blood clot, the RF wire may be removed from the artery and the catheter 914. After the blood clot is formed and the heat dissipation effect of the blood flow is terminated by the clot, a heated dilation balloon ablation method may be performed to locally heat and ablate the pulmonary artery, without fully collapsing the artery.

FIG. 21 illustrates the bronchus 910 having the adjacent pulmonary artery 912. A catheter 914 may deliver an arterial occlusion device 930 into a lumen 918 through the bronchus 910. The location of the pulmonary artery 912 relative to the bronchus 910 may be determined using, for example, intra-operative imaging. The catheter 914 may be rotated to the direction of the pulmonary artery 912 and the arterial occlusion device 930 may be advanced though the bronchial wall 920 into the pulmonary artery 912. In this embodiment, the arterial occlusion device may be a hollow needle catheter for delivery of an occlusive material 932. After placement in the pulmonary artery 912, the needle catheter may deliver the occlusive material into the artery 912 to create a clot. The occlusive material may be injected and may include a fibrin glue, cyanoacrylate, liquid comprising microspheres, alcohol, heated liquid such as water, collagen, tranexamic acid, thromboxane, adenosine diphosphate, and/or a gelatin sponge. The occlusive material may cause platelet aggregation resulting in a blood clot or the material itself may cause an occlusive clot. After the blood clot is formed and the heat dissipation effect of the blood flow is terminated by the clot, a heated dilation balloon ablation method may be performed to locally heat and ablate the pulmonary artery, without fully collapsing the artery.

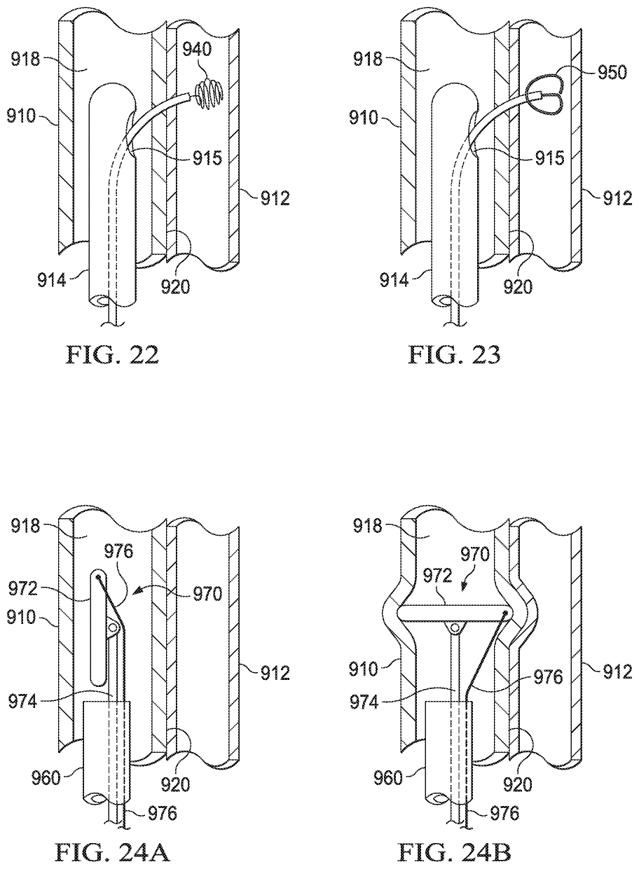

FIG. 22 illustrates the catheter 914 for delivery of an arterial occlusion device 940 into the lumen 918 of the bronchus 910. The location of the pulmonary artery 912 relative to the bronchus 910 may be determined using, for example, intra-operative imaging. The catheter 914 may be rotated to the direction of the pulmonary artery 912 and the arterial occlusion device 940 may be advanced though the bronchial wall 920 into the pulmonary artery 912. In this embodiment, the arterial occlusion device may include a delivery catheter for delivery of an occlusive coil. The coil may be formed of an elastic material, such as nitinol or an elastomer, that may be straightened for deployment through the delivery catheter and may return to a coiled shape after emerging from the delivery catheter and being inserted into the artery 912. The coil may cause platelet aggregation resulting in a blood clot. After the blood clot is formed and the heat dissipation effect of the blood flow is terminated by the clot, a heated dilation balloon ablation method may be performed to locally heat and ablate the pulmonary artery, without fully collapsing the artery.

FIG. 23 illustrates the catheter 914 for delivery of an arterial occlusion device 950 into the lumen 918 of the bronchus 910. The location of the pulmonary artery 912 relative to the bronchus 910 may be determined using, for example, intra-operative imaging. The catheter 914 may be rotated to the direction of the pulmonary artery 912 and the arterial occlusion device 940 may be advanced though the bronchial wall 920 into the pulmonary artery 912. In this embodiment, the arterial occlusion device may include a delivery catheter for delivery of a balloon. The balloon may be a silicone balloon extended over an opening in the delivery catheter. The balloon may be inflated with a liquid that has a high viscosity, such as a fibrin glue. Once inflated, the delivery catheter may be withdrawn from the balloon, leaving the balloon behind to occlude the artery. After the balloon is inflated and the heat dissipation effect of the blood flow is terminated by the balloon, a heated dilation balloon ablation method may be performed to locally heat and ablate the pulmonary artery, without fully collapsing the artery.

FIGS. 24A and 24B illustrate a catheter 960 for delivery of an arterial occlusion device 970 into the lumen 918 of the bronchus 910. In this embodiment, the arterial occlusion device 970 may include an occlusion bar 972 pivotally coupled to an RF rod 974. A pullwire 976 coupled to the occlusion bar 972 may be activated to pivot the occlusion bar from an insertion configuration parallel to the catheter 960 (as in FIG. 24A) to an occlusion configuration (as in FIG. 24B) in which the occlusion bar 972 is approximately transverse to the catheter 960. The occlusion bar 972 in the occlusion configuration may compress the artery 912 to fully or partially occlude blood flow through the artery. RF energy from the RF rod 974 may heat the occlusion bar 972 to cause platelet aggregation resulting in a blood clot in the compressed artery 912. After the blood clot is formed and the heat dissipation effect of the blood flow is terminated by the clot, a heated dilation balloon ablation method may be performed to further locally heat and ablate the pulmonary artery.

In some embodiments, the flow of air through a bronchial passageway may be blocked in addition to or as an alternative to blocking blood flow through the pulmonary artery. Ablation of the airway may induce neointimal hyperplasia and occlusion during the healing process. However, this process may take several days and may be too slow. In some embodiments, a physical airway occlusion device may be inserted after an airway heating procedure. The airway occlusion device may include a plug (e.g. a silicone or plastic plug), a one-way valve device, a glue and/or a foam. The plug may be temporarily placed and may be removed after intended necrosis is complete. Alternatively, it may be absorbed by the body or may be left permanently.

Any of the methods, techniques, or systems described in this disclosure may be used in combination or series with each other or with the methods, techniques, or systems described in the incorporated by reference PCT Application (Docket No. P02302-WO) filed Jul. 7, 2020, titled “Systems and Method for Localized Endoluminal Thermal Liquid Treatment.” Use of both the localized expandable device treatment and the diffuse liquid treatment may be useful, for example, in emphysema treatment when partial infarction is desirable. If insufficient heat energy can be delivered to ablate the bronchial arteries with the diffuse liquid method, the dilated heated balloon method may be used to create bronchial artery ablation after or prior to the diffuse method. Thus, a maximum number of airways may be occluded and partial infarction for additional lung volume reduction may be achieved. Similarly, combination treatments may be used for lung tumor ablation. Ablating the microvasculature of the segment in which the tumor resides via the diffuse method either before or after the balloon treatment method may ensure complete infarction of the segment.

In some embodiments, the systems and methods disclosed herein may be used in a medical procedure performed with a robot-assisted medical system as described in further detail below. As shown in FIG. 27, a robot-assisted medical system 1000 may include a manipulator assembly 1002 for operating a medical instrument 1004 (e.g., medical instrument system 100 or any of the medical instruments described above) in performing various procedures on a patient P positioned on a table T in a surgical environment 1001. The manipulator assembly 1002 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 1006, which may be inside or outside of the surgical environment 1001, generally includes one or more control devices for controlling manipulator assembly 1002. Manipulator assembly 1002 supports medical instrument 1004 and may optionally include a plurality of actuators or motors that drive inputs on medical instrument 1004 in response to commands from a control system 1012. The actuators may optionally include drive systems that when coupled to medical instrument 1004 may advance medical instrument 1004 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes).

Robot-assisted medical system 1000 also includes a display system 1010 for displaying an image or representation of the surgical site and medical instrument 1004 generated by a sensor system 1008 which may include an endoscopic imaging system. Display system 1010 and master assembly 1006 may be oriented so an operator O can control medical instrument 1004 and master assembly 1006 with the perception of telepresence.

The sensor system 1008 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 1004. The sensor system 1008 may also include temperature sensors.

Robot-assisted medical system 1000 may also include control system 1012. Control system 1012 includes at least one memory 1016 and at least one computer processor 1014 for effecting control between medical instrument 1004, master assembly 1006, sensor system 1008, and display system 1010. Control system 1012 also includes programmed instructions (e.g., a non-transitory machine-readable medium storing the instructions) to implement a plurality of operating modes of the robot-assisted medical system including a navigation planning mode, a navigation mode, and/or a procedure mode. Control system 1012 also includes programmed instructions (e.g., a non-transitory machine-readable medium storing the instructions) to implement some or all of the processes described in accordance with aspects disclosed herein, including, for example, expanding an expandable device, regulating the temperature of a heating system, controlling insertion and retraction of a treatment instrument, controlling actuation of a distal end of the treatment instrument, receiving sensor information, selecting a treatment location, deploying an occlusion device and/or determining a size of an anatomic lumen.

Control system 1012 may optionally further include a virtual visualization system to provide navigation assistance to operator O when controlling medical instrument 1004 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions. Not all the illustrated processes may be performed in all embodiments of the disclosed methods. Additionally, one or more processes that are not expressly illustrated in may be included before, after, in between, or as part of the illustrated processes. In some embodiments, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some embodiments are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in embodiments of this disclosure may be implemented in software to execute on a processor of a computer system such as control processing system. When implemented in software, the elements of the embodiments of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

Various aspects of the subject matter described herein are set forth in the following numbered examples.

Example 1: A method comprises positioning a catheter in an anatomic lumen, the catheter including a distal portion; deploying an occlusion device from the catheter; heating a liquid in the distal portion of the catheter to a temperature of less than 100 degrees Celsius with a heating device at the distal portion of the catheter; and releasing the heated liquid from the distal portion of the catheter into the anatomic lumen, wherein the occlusion device restricts flow of the heated liquid proximally of the occlusion device. The temperature may be less than the temperature at which the liquid becomes vapor. For fluids other than water at sea level pressure, a vaporization or boiling temperature may be greater than 100 degrees Celsius. For example, oil-based liquids may have a boiling point greater than 100 degrees Celsius.

Example 2: The method of Example 1, wherein the anatomic lumen is an airway in a lung.

Example 3: The method of Example 1 wherein the liquid is contained in a heated liquid reservoir coupled to the proximal portion of the catheter.

Example 4: A system comprising: a catheter sized to extend within a first endoluminal passageway, the catheter including a distal opening; and an occlusion device configured to extend through the catheter, through the distal opening, through a wall of the first endoluminal passageway and into a second endoluminal passageway adjacent to the first endoluminal passageway to generate an occlusion in the second endoluminal passageway.

Example 5: The system of Example 4, wherein the occlusion device includes an RF wire.

Example 6: The system of Example 5, wherein the occlusion device includes a hollow needle configured to deliver an occlusive material into the second endoluminal passageway.

Example 7: The system of Example 5, wherein the occlusion device includes a resistive coil.

Example 8: The system of Example 5, wherein the occlusion device includes a balloon catheter and a balloon coupled to a distal end of the balloon catheter.

Example 9: A system comprising a catheter sized to extend within a first endoluminal passageway, the catheter including a distal opening; and an occlusion device configured to extend from the distal opening, the occlusion device including a rod and an occlusion bar pivotally coupled to the rod, wherein in a first configuration the occlusion bar extends generally parallel to a longitudinal axis of the catheter and wherein in a second configuration the occlusion bar extends generally transverse to the longitudinal axis of the catheter and compresses a second endoluminal passageway adjacent to the first endoluminal passageway.

Example 10: The system of Example 9, wherein the rod in configured to transmit radiofrequency energy to the occlusion bar to heat the second endoluminal passageway.

Example 11: The system of Example 9, further comprising a pullwire coupled to the occlusion bar to transition the occlusion bar from the first configuration to the second configuration.

1. A system comprising:
a liquid source from which a liquid is delivered;
a catheter coupled to the liquid source, the catheter including a distal portion wherein the catheter is configured to release the liquid from the distal portion into an anatomic lumen;
an occlusion device coupled to the catheter and configured to prevent flow of the liquid in the anatomic lumen proximally of the occlusion device;
a computer processor;
a robot-assisted manipulator coupled to the catheter; and
a control system coupled to the computer processor,
wherein the control system is configured to actuate the robot-assisted manipulator to translate the catheter as the liquid is released from the distal portion into the anatomic lumen.

2-26. (canceled)

27. The system of claim 1, further comprising:
a heating device near the distal portion of the catheter, the heating device configured to heat the liquid to a temperature of less than a vaporization temperature for the liquid.

28. The system of claim 27, wherein the heating device is positioned within a lumen of the catheter at the distal portion of the catheter.

29. The system of claim 27, wherein the heating device includes a resistive coil, a resistive wire, an RF device, a microwave device, an ultrasound device, a high intensity focused ultrasound device, or an RF wire mesh.

30. The system of claim 27 wherein the heating device includes an optical fiber optically coupled to a light source, the optical fiber extending within the catheter.

31. The system of claim 27 wherein the heating device includes a magnetic field generator configured to excite a plurality of magnetic particles in the liquid.

32. The system of claim 1, wherein the occlusion device includes a slippery surface to allows translation of the catheter within the anatomic lumen.

33. The system of claim 1, wherein the control system is configured to at least partially collapse the occlusion device to allow for translation of the catheter within the anatomic lumen.

34. The system of claim 1 further comprising:
an outer catheter, wherein the catheter is an inner catheter received within the outer catheter and wherein the occlusion device is fixed to the outer catheter.

35. The system of claim 1, wherein the liquid is at least one of a saline liquid, a gel, sterile water, or a de-ionized water.

36. The system of claim 1, wherein the liquid includes a radiopaque material or an antimicrobial material.

37. The system of claim 1, further comprising a suction source configured to remove the liquid from the anatomic lumen.

38. The system of claim 1 further comprising a flush reservoir coupled to the catheter and configured to deliver a flushing medium to the occlusion device.

39. The system of claim 1 further comprising a heating coil to heat the liquid, wherein the heating coil extends within a length of the catheter proximal to the distal portion.

40. The system of claim 1 further comprising a visualization system including a fiber optic cable and lens to visualize a placement of the occlusion device.

41. The system of claim 40, wherein an outer surface of the catheter includes a groove sized to receive the fiber optic cable, wherein the fiber optic cable is housed within a visualization catheter.

42. The system of claim 1, wherein the computer processor is configured to receive information including a diseased state of the anatomic lumen.

43. The system of claim 42, wherein the computer processor is further configured to adjust at least one parameter based on the information.

44. The system of claim 43, wherein the at least one parameter includes temperature of the liquid, rate of release of the liquid, volume of released liquid, duration of liquid release, or pressure of the released liquid.

45. The system of claim 1, wherein the control system is configured to receive information about a size or a volume of the anatomic lumen and control a volume of the liquid released from the distal portion based on the received information about the size or volume of the anatomic lumen.
